(19) United States
(12) Patent Application Publication
(10) Pub. No.: US 2019/0159790 A1
(43) Pub. Date: May 30, 2019

(54) DENTAL DRILLING DEVICE

(71) Applicant: Nawakamon SURIYAN, Khlong Luang (TH)

(72) Inventor: Nawakamon SURIYAN, Khlong Luang (TH)

(21) Appl. No.: 15/755,016

(22) PCT Filed: Feb. 24, 2017
(86) PCT No.: PCT/TH2017/000015

§ 371 Date: Feb. 23, 2018

(51) Int. Cl. A61B 17/17; A61B 17/16; A61C 1/08; A61C 8/00

(52) U.S. Cl. CPC A61B 17/176; A61B 17/1637; A61B 2090/036; A61C 8/0089; A61C 1/084

(57) ABSTRACT

A dental drilling device includes a cutting unit and a direction control unit. The cutting unit has a tubular body providing a first end having a cutting end for harvesting a piece of bone and/or bone powder, and the second end coupled to a vertical stopper of the direction control unit. The tubular body has at least one opening for removing a piece of bone and/or bone powder. The direction control unit has a vertical stopper and a ring for establishing or limiting vertical and horizontal dimensions or borders of an implant site. A process or procedure for preparing an implant site by using the device includes affixing a ring to a surgical template for preparing a ring-affixed template suitable for forming an implant site; aligning a cutting end of dental drilling device within the ring; and cutting bone for forming or producing the implant site.

### TECHNICAL FIELD

The present disclosure relates to a dental drilling device. More particularly, the present disclosure relates to a dental drilling device for dental implants.

### BACKGROUND OF THE INVENTION

A conventional procedure for dental implantation begins with drilling a small hole which is further progressively enlarged in order to properly fit with a dental implant. In most cases, bone grafting is also required to stabilize an implant by replacing some missing bone. Because this procedure may require a synthetic or artificial bone graft to be applied with an implant, an attempt has been made to develop a drilling device which can prepare a wide range of osteotomy sizes corresponding to implant sizes, and simultaneously preserve an autologous bone to be used for grafting with dental implant procedures.

U.S. patent application Ser. No. 12/860,830 discloses the use of a guide pin system trephine drill for placing a dental implant and collecting an autogenous bone at the same time. Prior to cutting bone with a trephine, a first pilot drill having a diameter of 1-2 millimeters is operated without collecting bone and the trephine has been subsequently used for cutting and harvesting bone. Because of the structure of first pilot drill, the cutting edge of the trephine has a diameter of at least 4 millimeters. Therefore, the osteotomy size appears not to conform with implants having diameters of less than 4 millimeters.

U.S. patent application Ser. No. 13/056,560 discloses a trephine drill for implant procedures. The trephine drill has a perforating body for cutting alveolar bone and a rod body for adjusting a perforation depth. Having been internally occupied by the rod body, the trephine drill is able to prepare only larger osteotomies and may be displaceable during operating.

Therefore, there is a need to develop a dental drilling device that precisely controls a cutting direction in both horizontal and vertical dimensions, for preparing an implant site in a manner that is compatible with a wide range of implant diameters and which is capable of simultaneously harvesting an autologous bone graft.

### SUMMARY

An object of the present disclosure is to provide a dental drilling device. The device includes a cutting unit which has a cutting end for harvesting a bone, either in the form of pieces or powder, and a direction control unit (e.g., implemented as a set of vertical direction control elements or structures and a set of horizontal direction control elements or structures), which in several embodiments has a vertical stopper as a vertical direction control element or structure for limiting a vertical border of an implant site, plus a ring as a horizontal direction control element or structure, for also or further limiting a width of the implant site, resulting in the creation or provision of a precise, precisely formed, or precisely controlled space for dental implant preparation. Additionally, the device can have an opening for removing a penetrated bone.

Another object of the present disclosure is aimed at a technique or method for preparing an implant site by using a dental drilling device. Substantially, the method includes providing a dental drill device having a cutting unit and a direction control unit such as that described above; in several embodiments, affixing a ring, as a horizontal direction control, to a surgical template for preparing a ring-affixed template; aligning a cutting end of the cutting unit within the ring; and cutting bone for preparing an implant site.

### BRIEF DESCRIPTION OF THE DRAWINGS

FIG. 1 is a perspective view illustrating an embodiment of a dental drilling device having a longitudinal cut of the cutting end.

FIG. 2 is a perspective view illustrating an embodiment of a dental drilling device having multiple lateral openings.

FIG. 3 is a perspective view illustrating a tapered cut of the cutting end.

FIG. 4A is a perspective view illustrating a ring.

FIG. 4B is a side elevational view illustrating a ring.

FIG. 5 is a perspective view illustrating a ring-affixed template.

### DETAILED DESCRIPTION

As used herein, the term “set” means one or more, i.e., at least one.

The term “implant” or “dental implant” as used herein refers to a dental fixture or a surgical component that interfaces with or which is insertable or inserted into maxillary or mandible bones for supporting a dental prosthesis.

The term “vertical” as used herein refers to a direction or trajectory along which a depth measure or value, such a current instrument or device depth level in an osteotomy, can vary. Such variation can occur relative to a maximum depth such as the overall depth of the osteotomy (e.g., as a result of deeper entry of the instrument or device into the osteotomy, or progressive withdrawal of the instrument or device from the osteotomy). Moreover, in a dental context, vertical can correspond to or be defined as a direction extending along, parallel, or generally parallel to a superior or inferior anatomical direction, particularly with respect to the alveolar process.

The term “horizontal” as used herein refers to a direction or trajectory within a plane perpendicular to a vertical axis or center line extending through the depth of an osteotomy, for instance, radially toward or away from such a vertical axis or center line between bone surfaces that form the lateral borders of the osteotomy. Moreover, in a dental context, horizontal can refer to a direction extending along, parallel, or generally parallel to a medial or lateral anatomical direction.

The term “implant site” or “an implant site” refers to a recess or recessed hole that forms an osteotomy into which a dental implant is insertable or inserted. In addition, “implant site” can be defined to include a cross-sectional upper or top surface area or implant entry region, a lower or bottom surface area, and a set of sidewalls that extend therebetween, forming the osteotomy into which a dental implant is insertable or inserted. Also, “implant site” can include, define, or refer to a surface location or a predetermined area or region at which an osteotomy is formed or prepared for insertion of a dental implant therein.

Hereafter, representative embodiments which are described in description and drawings are not limited, but are proposed as representative examples for purpose of clarity and understanding. It is envisioned that a person having an ordinary skill in the art can carry out various modifications to the representative embodiments described herein without departing from the scope of the present disclosure and the corresponding invention that is defined in the appended claims.

### Aspects of a Dental Drilling Device

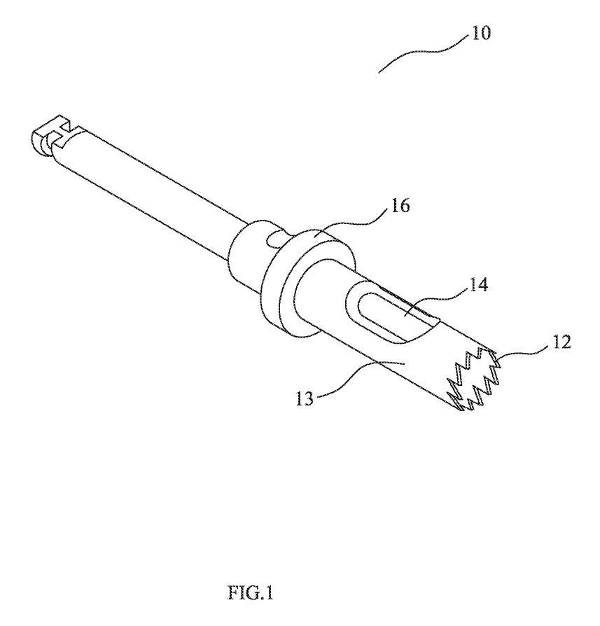

The presently-disclosed representative embodiments, detailed herein with respect to FIGS. 1-5, describe a dental drilling device 10 having a cutting unit for penetrating bone, and a direction control unit for controlling, guiding, and/or limiting the movement directions or range or extent of directional motion of the cutting unit, in both vertical and horizontal dimensions or directions.

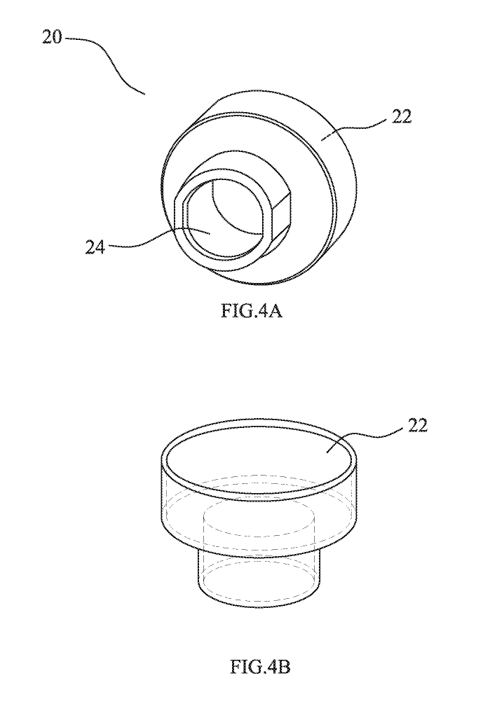

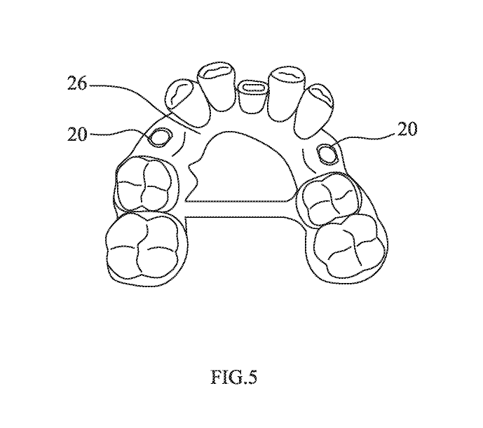

In various embodiments, the direction control unit includes or is implemented as a vertical stopper 16 as shown in FIGS. 1-3, and a ring 20 as shown in FIGS. 4A, 4B, and 5, for respectively establishing controlling, guiding, and/or limiting the range or extent of vertical and horizontal motion of the cutting unit. The cutting unit includes a tubular body 13 having a first end and a second end. The first end provides a cutting end or cutting structure for penetrating bone, whereas the second end is coupled to the vertical stopper 16. Additionally, the tubular body 13 further includes an opening 14 for removing portions of a penetrated bone. Regarding the direction control unit, as shown in FIGS. 4A, 4B, and 5 the ring 20 typically has a first portion 22 and a second portion 24 for mating or assembling with the vertical stopper 16 and the cutting end, respectively. As such, embodiments in accordance with the present disclosure are able to precisely create or prepare an adequate space for a dental implant, together with collecting portions of a penetrated bone so that dental prosthetic(s) can be precisely and properly implanted with an autologous bone graft, if desired or needed.

With reference again to FIGS. 1-3, in accordance with various embodiments the tubular body 13 of the cutting unit has a first end and a second end. The first end is designed for penetrating bone by having a cutting end configured for preparing or forming an implant site that precisely provides adequate space for a dental implant and harvesting bone, in association with or during implant site preparation or formation. In general application, a dental implant typically has a diameter of 2-6 millimeters. The dental drilling device 10 is capable of osteotomy preparation at various diameters or diameter ranges, particularly a selected or predetermined diameter corresponding to the diameter of the dental implant, typically or preferably 2-6 millimeters. Particularly, the device 10, in absence of a central guide for preparing a pilot hole, is able to prepare a small implant site for a dental implant as small 2-millimete in size or diameter. To prepare a precise cut and hence a precisely defined implant site for dental implantation, the direction control unit is configured for establishing or limiting the extent or range of vertical and horizontal directional cutting unit motion. Having a penetrating function, the cutting end may be shaped in a manner that corresponds to or which provides a longitudinal cut 12 and/or a tapered cut 11. One or both types of cuts can be applicable depending upon design details, such as designs that include saw-like, serrated, diamond-coated, sharp-edged, pointed teeth, rounded teeth, and/or the like. A bone specimen or sample which is collectable by using the longitudinal cut and the tapered cut is in form of a piece of bone and bone powder, respectively. With regard to the opposite or other side of the tubular body 13, the second end is coupled to the vertical stopper 16 to precisely limit the depth of bone penetration.

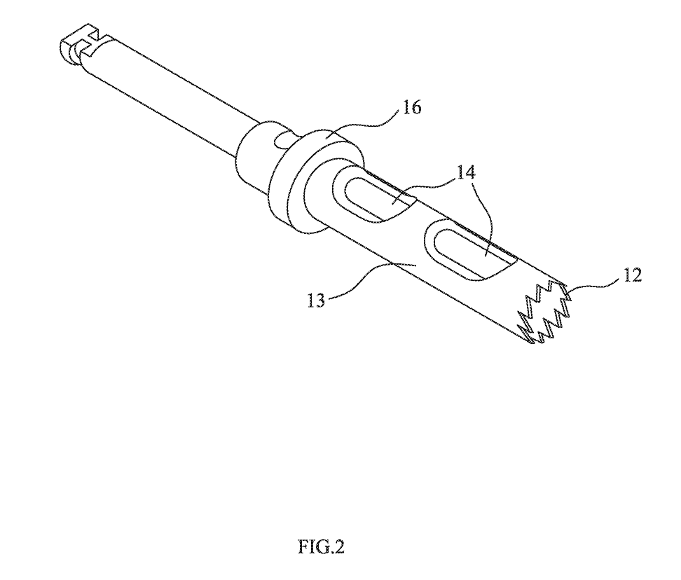

FIGS. 1 and 2 illustrate particular representative embodiments of a dental drilling device 10 configured for harvesting one or more bone pieces during implant site formation in accordance with the present disclosure. Such a device 10 has a longitudinal cut or longitudinal cutting structure 12 at a first end of the tubular body 13, while a second end of the tubular body 13 is coupled to the vertical stopper 16. During use, the longitudinal cut 12 is alignable within the ring 20 shown in FIGS. 4A, 4B, and 5 for providing, limiting, or precisely controlling the horizontal extent of bone cutting. A piece, portion, specimen, or sample of penetrated bone is harvested, and can be used for autologous bone grafting in a manner readily understood by individuals having ordinary skill in the relevant art. As indicated in FIGS. 1 and 2, the tubular body 13 has at least one lateral opening 14 for enabling removal of at least one piece, portion, specimen, or sample of penetrated bone.

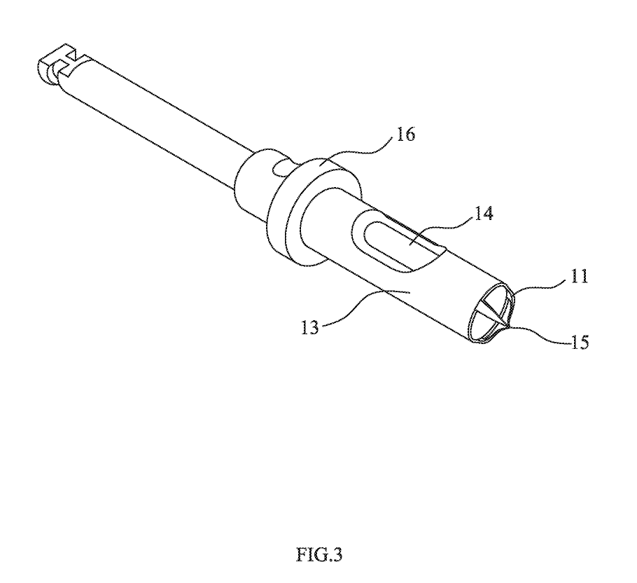

FIG. 3 illustrates another representative embodiment of a dental drilling device 10. The device 10 has a tapered cut or tapered cutting structure 11 at the first end of the tubular body 13. During use, the alignment or relative positioning of the tapered cut 11 with respect to or in the ring 20 precisely establishes or limits a horizontal border of an implant site. The cutting areas of the tapered cut 11 include central 15 and radial or radius areas. However, when this embodiment is used or in operation, the portion, sample, or specimen of penetrated bone, which is produced from the central and radius areas of the tapered cut 11, is at least primarily in form of bone powder, rather than primarily in the form of a contiguous piece of bone. This bone powder as such is collectable into the tubular body 13, and is usable for autologous bone grafting during an implant procedure.

In accordance with some embodiments, the tubular body 13 further includes an opening 14, particularly a lateral opening, which can be located along or at a lateral wall of the tubular body 13, for removing bone which is cut by the cutting end, e.g., the tapered cut 11. In a manner essentially identical or analogous to that shown in FIG. 2, the tubular body 13 can be designed to have more than one opening 14, e.g., multiple lateral openings spaced lengthwise or longitudinally along portions of the tubular body 13. A typically desirable number of openings 14 is between 1-4. Unlike conventional procedures which may utilize a drill for gradual enlargement of bony space without bone collecting, a penetrated portion of bone in accordance with various embodiments of the present disclosure is collectable inside the tubular body 13, and then removable via a set of openings 14 for functional usage of an autologous graft during dental implantation. As a result, the use of synthetic or artificial bone is not required, and is optional, negligible, unnecessary, or eliminated when an embodiment in accordance with the present disclosure is utilized for an implant procedure.

In accordance with several embodiments, the vertical stopper 16 is used for precisely establishing or limiting a vertical border or depth of an implant site. For controlling the depth of a dental implant, it can be very helpful to make a calculation of implantation parameters beforehand, such as a depth, a width, and an angulation of each implant site, by using either computing software, tomographic imaging, radiographic imaging, or 3-D imaging, or the like in a manner readily understood by individuals having ordinary skill in the relevant art, which aids or enables the selection of a vertical stopper 16 having an appropriate corresponding length or height for each case or implant site under consideration. In multiple representative embodiments, the length of the device 10 has a range of 6-14 millimeters, such that the length from the cutting end to the vertical stopper 16 is compatible or appropriately matched with the predetermined depth of the implant site.

In accordance with various embodiments, the ring 20 is configured for precisely establishing or limiting the horizontal border, cross-sectional area, span, extent, diameter or width of an implant site. For dental reconstruction with implant, it is required to individually utilize different sizes of implants for various sites (such as anterior or posterior, mandible or maxillary sites or areas). Implants having diameters of 2-6 millimeters are commonly used in such procedures, in a manner readily understood by individuals having ordinary skill in the relevant art. Sizes of the ring 20, the cutting end, and a given dental implant should be correspondingly matched for precisely establishing correct and secure placement of dental implant. More specifically, in various embodiments the ring 20 has a first portion 22 and a second portion 24, where the first portion 22 is extended to form the second portion 24 and an inner diameter of the first portion 22 is relatively larger than that of the second portion 24. For example, inner diameters of the first and second portions 22, 24 can be 5.77 and 3.77 millimeters, for mating or assembling with diameters of the vertical stopper 16 and the cutting end which are 5.75 and 3.75 millimeters, respectively.

When the device 10 is used, the cutting unit is inserted into or assembled with the ring 20. More particularly, the cutting end is passed through the first portion 22 of the ring 20, and then engaged with or within the second portion 24 of the ring for penetrating into a bone, while the vertical stopper 16, which is coupled to the second end of the cutting unit, is retainable within the first portion 22 of the ring 20 which has a relatively larger diameter than that of the second portion 24 of the ring for precisely limiting the vertical extent or dimension of the implant site. As a result, the vertical depth of the implant site, i.e, the osteotomy under consideration at which the dental implant under consideration will be implanted, is precisely controlled.

FIG. 5 illustrates a representative ring 20 affixed into a representative surgical template to thereby provide a ring-affixed 26 in accordance with an embodiment of the present disclosure. For placement of multiple dental implants, multiple rings 20 may be prepared to apply a surgical template during a single visit. Moreover, a second portion 24 of the ring 20 is close to bone or a bony part, whereas a first portion 22 is relatively distal to bone or a bony part for retaining the vertical stopper 16.

For the direction control unit, the cutting end of the tubular body 13 is aligned within the ring 20 for horizontal demarcation, while the device 10 is vertically perforating into a bone until achieving a precise predetermined maximum depth by way of vertical stopper control. Taken together, the vertical stopper 16 and the ring 20 ensure that an implant site osteotomy exhibits precise dimensions in depth and width. In this regard, the selected dental implant can be precisely and properly placed into the osteotomy site with autologous bone graft, if needed.

### Aspects of a Technique or Method for Preparing an Implant Site

In accordance with a number of embodiments, the present disclosure relates to a technique, process, procedure, or method for preparing or forming an implant site by using a dental drilling device 10 such as described herein. Substantially, the technique, process, procedure, or method includes: providing a dental drilling device 10 having a cutting unit and a direction control unit as described above; affixing a ring 20 to a surgical template for preparing or generating a ring-affixed template 26; aligning a cutting end of the cutting unit within the ring 20; and cutting bone while preparing or forming the implant site.

In accordance with a number of embodiments, a dental drilling device 10 is provided for preparing or forming an implant site for a dental implant procedure. The dental drilling device 10 includes a cutting unit and a direction control unit. The cutting unit has a tubular body 13, which includes a first end having a cutting end for penetrating bone, and a second end coupled to a vertical stopper 16 of the direction control unit. The direction control unit includes a vertical stopper 16 and a ring 20 for precisely controlling the range of motion of the cutting unit in both vertical and horizontal directions or dimensions, respectively. The ring 20 has a first portion 22 and a second portion 24, wherein a cross-sectional area or diameter of the former is relatively larger than that of the latter. Additionally, the tubular body 13 further includes at least one opening 14 for removing one or more pieces, samples, or specimens of penetrated bone or bone powder.

In accordance with most embodiments, after details of implant sites and sizes are reasonably decided, a ring 20 and a cutting unit are correspondingly selected and then a surgical template identical to the dental anatomy of a patient is prepared. The ring 20 is subsequently affixed into the surgical template for preparing a ring-affixed template 26. For some patients who need multiple implants treatment, multiple rings 20 are affixed into the surgical template to create the ring-affixed template 26. For example, the attachment of the ring(s) 20 to the surgical template can occur by drilling a hole into the template and then placing the ring 20 into the hole. Alternatively, the ring 20 can be glued or cemented to the surgical template. The ring-affixed template 26 can be positioned or adjusted relative to a set of implant sites. A second portion 24 of the ring 20 is closely positioned or placed relative to a bony site, whereas a first portion 22 of the ring 20, a larger portion in terms of its cross-sectional area or diameter, is distally located relative to each the bony site and the first portion 22 of the ring 20 for receiving or engaging with the vertical stopper 16.

In accordance with various embodiments, a dental drilling device 10, which mainly includes a cutting unit and a direction control unit, is applied to the ring-affixed template 26. More specifically, a cutting end of a cutting unit is aligned relative to the ring 20 or a given ring 20 under consideration, which has been already affixed to the ring-affixed template 26. This is intended to limit a horizontal border of the implant site, i.e. the osteotomy formed. The cutting end, either in the form of a longitudinal cut 12 or a tapered cut 11, of the dental drilling device 10 is passed from the ring's first portion 22 through the second portion 24, and is vertically limited by a vertical stopper 16, which is retainable in the first portion 22 of the ring 20 that receives or engages with the vertical stopper 16.

In accordance with multiple embodiments, the dental drilling device 10 with proper alignment cuts into a bone until the vertical stopper 16 is blocked by the first portion 22 of the ring 20. As a result, a bony structure corresponding to an implant size under consideration (e.g., a selected or predetermined implant size) is precisely penetrated in diameter and length. Additionally, penetrated bone, either in form of a piece of bone or bone powder, can be removed via an opening 14 of the tubular body and used as an autologous bone graft for bone augmentation. In some embodiments, multiple openings are present and are suitably designed or provided at a lateral wall of the tubular body.

In view of the foregoing, embodiments in accordance with the present disclosure are configured for enabling precise implant site preparation or formation, even for small osteotomy and corresponding dental implant sizes (e.g. 2 millimeters in diameter), without losing penetrated bone material. The penetrated bone material, which is efficiently harvested by embodiments in accordance with the present disclosure, can be reserved or removed for one or more autologous bone grafts, resulting in an operationally effective and cost saving procedure.

1. A dental drilling device comprising:
a cutting unit having a tubular body which has a first end and a second end, wherein the first end has a cutting end for penetrating bone; and
a direction control unit comprising a vertical stopper coupled to the second end for limiting a vertical border of an implant site and a ring having a first portion extending to a second portion for limiting a horizontal border of the implant site,
wherein the cutting end is guided by the direction control unit.

2. (canceled)

3. The dental drilling device according to claim 1, wherein the first portion has an inner diameter relatively larger than that of the second portion.

4. The dental drilling device according to claim 1, wherein the cutting end is aligned within the second portion of the ring.

5. The dental drilling device according to claim 1, wherein a diameter of the cutting end corresponds to that of the second portion.

6. The dental drilling device according to claim 1, wherein the cutting end is selected from a longitudinal cut and a tapered cut.

7. The dental drilling device according to claim 1, wherein the cutting end has a diameter of 2-6 millimeters.

8. The dental drilling device according to claim 1, wherein the cutting end is passed through the first portion for entering into the second portion and the vertical stopper is retainable within the second portion for limiting the vertical border of the implant site.

9. The dental drilling device according to claim 1, wherein the tubular body further comprises at least one opening for removing a piece of bone and/or bone powder.

10. The dental drilling device according to claim 9, wherein the number of openings is 1-4.

11. The dental drilling device according to claim 9, wherein each opening is located along a lateral wall of the tubular body.

12. A method for preparing an implant site, the method comprising:
providing a dental drilling device comprising
a cutting unit having a tubular body, wherein the tubular body has a first end having a cutting end for harvesting a piece of bone and/or bone powder and a second end; and
a direction control unit comprising a vertical stopper coupled to the second end of the tubular body for limiting a vertical border and a ring having a first portion for receiving the vertical stopper and a second portion for limiting a horizontal border of an implant;
affixing the ring to a surgical template for preparing a ring-affixed template;
aligning the cutting end within the second portion of the ring; and
cutting the bone to form an osteotomy corresponding to an implant size.

13. The method according to claim 12 further comprising removing the piece of bone and/or the bone powder via an opening of the tubular body.

14. The method according to claim 12, wherein the implant size has a diameter of 2-6 millimeters.

15. The dental drilling device according to claim 3, wherein the first portion has an inner diameter relatively larger than that of the second portion.

16. The dental drilling device according to claim 4, wherein the first portion has an inner diameter relatively larger than that of the second portion.

17. The dental drilling device according to claim 10, wherein each opening is located along a lateral wall of the tubular body.

18. The method according to claim 13, wherein the implant size has a diameter of 2-6 millimeters.
